(19) United States
(12) Patent Application Publication
(10) Pub. No.: US 2008/0287718 A1
(43) Pub. Date: Nov. 20, 2008

(54) Catalytically Active Porous Membrane Reactor for Reacting Organic Compounds

(75) Inventors: Aurel Wolf, Wulfrath (DE); Rafael Warsitz, Essen (DE); Andreas Nickel, Wetter (DE); Olaf Stange, Saulheim (DE); Reinhard Schomacker, Berlin (DE); Herry Purnama, Berlin (DE); Andrea Schmidt, Berlin (DE); Roland Dittmeyer, Oberursel (DE); Daniel Urbanczyk, Velbert (DE); Ingolf Voigt, Jena (DE)

Correspondence Address: NORRIS, MCLAUGHLIN & MARCUS, P.A., 875 THIRD AVE, 18TH FLOOR, NEW YORK, NY, 10022, US

(73) Assignee: BAYER TECHNOLOGY SERVICES GMBH, LEVERKUSEN (DE)

(21) Appl. No.: 11/817,425

(22) PCT Filed: Mar. 2, 2006
(86) PCT No.: PCT/EP2006/001893

§ 371 Date: Jun. 26, 2008

(30) Foreign Application Priority Data
Mar. 5, 2005 (DE) 10 2005 010 213.1

(51) Int. Cl. B01J 35/00; C07C 5/02; C07C 5/03

(52) U.S. Cl. 585/266; 502/4; 585/250

(57) ABSTRACT

The invention relates to a catalytically active membrane pore flow reactor, to the membrane used and to processes using this reactor.

The invention relates to a catalytically active membrane pore flow reactor, to the membrane used and to processes using this reactor.

Hydrogenations of organic substances are performed in industry with various types of reactors. The reactors can be divided roughly into fixed bed reactors and suspension reactors. In catalytic hydrogenation reactions, the trickle film reactor is of the greatest industrial significance (Al-Dahhan et al. Ind. Engng Chem. Res. 36 (1997) 3292-3314, Saroha et al. Rev. Chem. Engng 12 (996) 207). In order to reduce the mass transfer limitation caused by pore diffusion in the catalyst particle, coated catalysts are used. A significant advantage in the case of use of a trickle film reactor is that the separation between the reaction solution and the catalyst after the reaction is not necessary. Moreover, the reactor can be operated continuously. Another industrially used reactor type for triphasic hydrogenation is the slurry reactor. Owing to its simplicity of construction, the simpler operation and the great flexibility, the slurry reactor is very often used for hydrogenation reactions on the industrial scale. Bubble column reactors are likewise frequently used, particularly in the field of organic synthesis, for example oxidation, chlorination, hydrogenation. The development of new reactor types for triphasic reactions, for example membrane reactors, is being researched intensively. Kuzin et al. (Kuzin et al., Catalysis Today 79 (2003) 105-111) describe the use of membrane reactors for the hydrogenation of organic compounds. The membrane, which consists for the most part of nickel, functions firstly as a support and secondly as a medium for the encounter of gas and liquid. De Vos (de Vos et al. Chem. Eng. Sci. 37 (1982) 1719) reports the use of ceramic membranes for a strongly exothermic reaction.

In triphasic hydrogenations, the low solubility of the hydrogen in the liquid phase generally constitutes a mass transfer limitation. Cini and Harold (Cini, Harold, AIChE Journal 37 (1991) 997-1008) describe a catalytic membrane reactor according to the diffusor principle. Compared to suspension catalysts, it was thus possible to achieve a rise in the reaction rate by the factor of 20. The cylindrical membrane consists of macroporous and microporous ceramic material. Another type of membrane reactor is the so-called catalytically active membrane flow reactor. In membrane flow reactors, the mass transfer can be improved significantly. This then leads to an increase in the reactor performance and a rise in the selectivity.

Golman et al. (Golman et al. J. Chem. Eng. Jpn. 30 (1997) 507-513) were able to show that the yield and the selectivity of a desired intermediate depend greatly on the membrane properties, the catalytic activity of the membrane and on the convective transport through the membrane. A reaction in which the mass is transferred predominantly by convection enables the full exploitation of the surface of the catalyst. Establishment of convective transport through the porous catalytically active membrane thus allows complete reaction control with regard to subsequent reactions to be achieved.

In the published literature, there is a series of publications which mention a membrane flow reactor. WO A 98/10865 discloses a membrane flow reactor having an amorphous microporous membrane with pore sizes of 0.5-2 nm. The aim of this membrane was the suppression of subsequent reactions by the prevention of backmixing owing to pore sizes in (double the) molecule size. However, such a membrane reactor has a very high pressure drop owing to the very small pore sizes, such that industrial scale operation would be uneconomic. U.S. Pat. No. 5,492,873 claims a membrane reactor with a membrane which is, however, permeable only to one reactant and not to the other reactants and catalyst poisons. This prevented catalyst poisoning. The reaction zone in such an arrangement arises merely through the surface, such that the catalyst utilization and the space-time yield are very low.

RU A 2083540 describes the performance of the hydrogenation reactions, in which the organic substance is saturated with hydrogen in a separate stirred tank and then the solution is passed through an external bed. Although this reactor concept utilizes the principle of presaturation of the organic solution, no overcoming of the internal mass transfer limitation is achieved here. In spite of the improvements to various types of industrially used reactors for the hydrogenation of unsaturated organic substances, the performance of conventional reactors, for example fixed beds or trickle beds, is still well below the performance of the slurry reactor with intrinsic kinetic measurements (Meile et al., Ind. Eng. Chem. Res. 41 (2002) 1711-1715). This indicates that the mass transfer limitation in such reactors is still present to a significant degree. In other words, the high mass transfer limitation then leads to ineffective exploitation of the catalysts. Moreover, such reactor systems are subject to constant deactivation, such that only short lifetimes are enabled. It was therefore an object of the present invention, proceeding from the prior art, to provide a so-called catalytic membrane pore flow reactor with which, inter alia, it becomes possible to perform hydrogenation reactions while ruling out mass transfer limitation, and with significantly prolonged lifetimes. The reactor performance of this membrane pore flow reactor should correspond to or exceed the performance of conventional reactors.

It has now been found that, surprisingly, a catalytic membrane pore flow reactor exhibits a higher space-time yield compared to other conventional reactors when, as a result of establishment of a sufficiently high convective volume flow through the membrane, all catalytically active particles come into contact optimally with reaction solution.

The invention thus provides a catalytically active membrane pore flow reactor for conversion, especially hydrogenation, of organic compounds.

The inventive reactor comprises the use of ceramic membranes consisting of Al₂O₃, TiO₂, ZrO₂, SiO₂ and other known ceramic membranes, for example MgAl₂O₄ and SiC, or consisting of binary and ternary mixtures of these materials, with different pore diameters. The pore diameter has a crucial role for the optimal (and inexpensive) performance of hydrogenations. Optimally, the pore diameter of the membrane has to be in the order of magnitude of the pores of catalysts in piece form. Accordingly, membranes with pore diameters in the range of 0.1 μm-100 μm, preferably in the range of 0.1 μm-50 μm and very preferably in the range of 0.1 μm-10 μm are used. Significantly smaller pores lead to a pressure drop and thus limit the amount which can be conveyed through the membrane. Excessively large pores lead subsequently to a limitation of diffusion.

The optimal residence time in the membrane pores to be established for the processes is from 1*10⁻⁶ to 5 s, preferably from 1*10⁻⁵ to 3 s and very preferably from 1*10⁻⁴ to 1 s. The flow rates in the pores needed for this purpose are in the range of 0-1 m's, preferably in the range of 1*10⁻³ to 0.1 m's. The residence times can be determined via the volume flow rate and the membrane geometry (membrane area, pore diameter and porosity) by means of methods commonly known to those skilled in the art (see E. Fitzer, W. Fritz, Technische Chemie [Industrial chemistry], 3rd Edition 1989, p. 45 and p. 277, Springer Verlag, or VDI-Wärmeatlas, Berechnungsblätter für den Wärmeübergang [Calculation sheets for heat transfer], series: VDI-Buch, VDI-Gesellschaft Verfahrenstechnik und Chemieingenieurwesen (GVC), (eds.), 9th, revised and extended edition, 2002, XIII, Chapter L “Druckverlust” [“Pressure drop”]). The ceramic membranes are first coated with a catalytic component. Useful components are all hydrogenation-active transition metals, for example Pd, Pt, Ni, Ru, Rh, etc. Drying, calcining and reduction are further conditioning steps which are used here as they are also used typically to activate the catalytic membrane. The complexity of the preparation of the inventive catalytically active membranes by coating is significantly less than the preparation of shell catalysts.

After successful preparation, so-called catalytically active pore flow membranes are obtained, which are in turn clamped into a metallic membrane module. The combination of catalytically active pore flow membrane and membrane module describes the membrane pore flow reactor, which is attached to the further plant periphery.

Useful reactive substrates include all organic compounds which have a hydrogenation-active functional group. This class includes, for example, C—C double bonds, C—C triple bonds, aromatic rings, carbonyl groups, nitrile groups, diolefins, etc. In principle, it would be possible to perform all heterogeneously catalyzed gas-liquid reactions, oxidations, alkylations, chlorinations, etc. in such a membrane pore flow reactor.

Useful organic solvents generally include all customary organic, protic and aprotic solvents, for example unsubstituted or substituted aromatic or nonaromatic hydrocarbons with an alkyl radical or halogen as a substituent, preferably haloalkanes, alcohols, water, ethers, haloaromatics, etc. Particular preference is given to hexane, methylcyclohexane, heptane, cumene, toluene, chlorobenzene, ethanol, isopropanol, water.

The temperature at which the hydrogenation is performed is limited by safety aspects and/or kinetic aspects. For example, such hydrogenations are performed in the temperature range of 20-300° C., preferably in the range of 40-250° C.

The hydrogen pressure of the performance of the hydrogenation is generally determined by kinetic and safety limits. Typically, but without being restricted to this range, hydrogenations proceed in the range of 1-300 bar.

With regard to the performance of the reaction, the procedure is typically such that the reactants (1) are introduced into an incorporated reservoir vessel (2). In this vessel, the reactants are saturated with hydrogen (3) by means of a sparging stirrer (4). However, the process is not restricted to this stirrer type but rather can be performed with all sparging units (stirrers, nozzles, etc.) known to those skilled in the art. The saturated liquid phase is passed with the aid of a pump (5) into the membrane pore flow reactor (6). There, the saturated reactant solution flows through the catalytically active pore flow membrane, where it reacts over the catalytically active reaction sites. The reaction mixture which subsequently leaves the membrane pore flow reactor (6) is recycled via a heat exchanger (7) into the reservoir vessel (2) or converted continuously in a cascade.

The throughput of the liquid phases is in the range from 20 to 500 ml/min, preferably in the range from 100 to 300 ml/min.

Surprisingly and advantageously, it is possible in the inventive reaction, through the skilful adjustment of the volume flow depending on the membrane pore structure, i.e. establishment of the residence time optimal for a membrane geometry, to enhance the conversion rate (space-time yield), such that the diffusion limitation which typically occurs can be overcome. This allows the achievement of conversion rates which are significantly higher than those of the conventional reactors, or, as a result of the further increase in the flow rate, in the limiting case, the conversion rate corresponding to the intrinsic kinetics is achieved.

Surprisingly, significantly longer lifetimes than with conventional reactors are achieved. This is suspected to be achieved as a result of the convective transport through the membrane, which constantly flows around the active catalytic sites, such that there is no deposition of reactants and secondary components in the convective region and, after a short initial phase, “no” deactivation occurs.

Surprisingly and advantageously, with this reactor type, compared to conventional reactor types (slurry, fixed bed), significantly higher selectivities are also achieved in hydrogenations with subsequent reactions.

The process according to the invention is notable for the high performance of the catalytically active membrane pore flow reactor, which, as a consequence, leads to greatly reduced reaction times in combination with significantly increased lifetimes. The reduction in the mass transfer limitation in the membrane pore flow reactor leads to an increase in the effective exploitation of the catalysts. Further advantages of the invention are as follows.

In processes with high conversion rates, it is possible for safety purposes to exert control through the flow rate, since it is directly proportional to the conversion rate. Owing to its apparatus simplicity and the uncomplicated experimental procedure, hydrogenation in the membrane flow reactor in particular is found to be a very advantageous process.

The examples which follow are intended to illustrate the present invention but without restricting it.

### EXAMPLES

### General

The tubular membranes of Al₂O₃ used in the process according to the invention have a length of 250 mm. The external diameter is 2.9 mm and the internal diameter 1.9 mm. The membranes have a mean weight of 2.9 g and their pore size is in the range of 3.0 μm to 0.6 μm. The proportion of the reactants used is in the range from 5 to 100% by volume, preferably in the range from 5 to 50% by volume.

### Membrane Preparation

The coating of the ceramic membranes was performed by means of chemical wet impregnation. The membranes were impregnated with a saturated palladium(II) acetate solution. The solvent used was toluene, since Pd(OAc)₂ has a satisfactory solubility in toluene. The saturation concentration of palladium(II) acetate in toluene at room temperature and atmospheric pressure was determined experimentally to be 10.75 gl⁻¹.

In the chemical wet impregnation, two variants have been tested. Both were effected at room temperature and atmospheric pressure. In the first case, the membrane to be coated had been immersed into a saturate palladium(II) acetate toluene solution at rest for several days. In the other case, the ceramic membrane immersed into a palladium solution was placed on a pivoting table for several hours. Subsequently, the membranes impregnated in Pd(OAc)₂ were dried under air for several hours. Calcination of the palladium in the porous ceramic membrane was dispensed with. In order to obtain metallic and hence catalytically active palladium, reduction was effected in a hydrogen stream. To this end, the impregnated membrane is placed in the membrane module and flowed through with hydrogen in the membrane flow reactor at 70° C. and p(H₂)=0.3 bar gauge pressure.

### Hydrogenation Procedure

The construction of the membrane flow reactor is shown in the figure which follows in the form of a process flow diagram. The catalytic hydrogenation of α-methylstyrene to cumene is effected batchwise according to the principle of a loop reactor. The membrane flow reactor is thus operated as a differential circulation reactor.

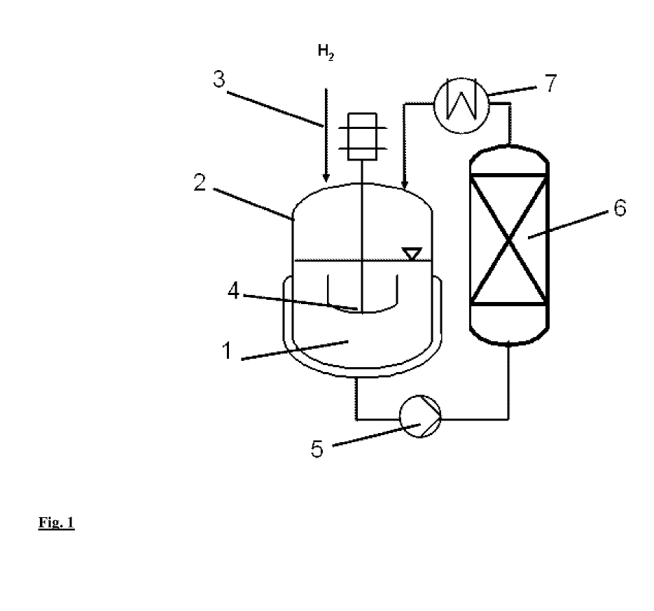

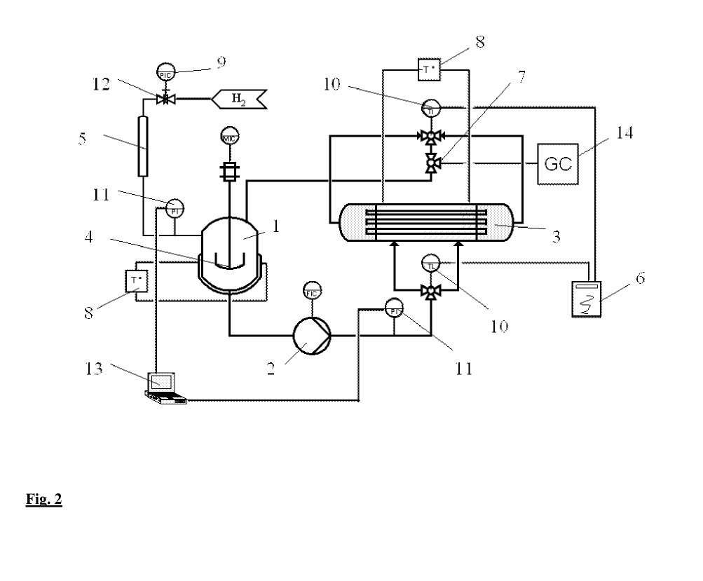

### REFERENCE NUMERALS FOR FIG. 2

### Construction of the Membrane Flow Reactor

(1) Saturation vessel
(2) Pump
(3) Membrane pore flow reactor with three catalytically active pore flow membranes
(4) Sparging stirrer
(5) Hydrogen reservoir
(6) Temperature plotter
(7) Valve for sampling
(8) Thermostat
(9) PID regulator
(10) Temperature sensor
(11) Pressure transducer (0-60 bar)
(12) Hydrogen shutoff valve
(13) Computer for online recording of the pressure
(14) Gas chromatograph

The characteristic feature of the experimental arrangement of the loop reactor is the spatial separation of the catalytic chemical reaction in the membrane and the saturation of the liquid phase with hydrogen.

In the reservoir vessel, the liquid phase is saturated with hydrogen with the aid of a sparging stirrer. Compared to conventional stirrer systems, sparging stirrers exhibit much higher mass transfer rates. Beyond the 45° slopes of the propeller, in the case of optimal rotational speed, owing to centrifugal forces, a reduced pressure arises, which results in an enormous suction force. Hydrogen from the gas space is introduced into the liquid medium via a hollow shaft of the stirrer. On the sparging stirrer, the stirrer speed can be adjusted and the relative torque read off.

From the reservoir vessel (1), the hydrogen-saturated solution is pumped by means of a pump (2) into the membrane pore flow reactor (3). In such a reactor, it is possible to position a maximum of three catalytically active pore flow membranes which are sealed with O rings made from Viton. The arrangement of the two reactor inlets and of the two reactor outlets can be exchanged with one another, such that the flow through the tubular pore flow membranes (from the inside outward or from the outside inward) can be varied. From the membrane pore flow reactor, the reaction solution is passed back to the saturation vessel.

Both the saturation vessel and the membrane module can be heated independently of one another. For this purpose, the reaction is embedded into an electrical heatable aluminum block. The saturation vessel is surrounded by a tube coil and is heated by means of a thermostat. The temperature is recorded by means of temperature sensors, in each case at the inlet and outlet of the membrane module.

The pressure is indicated by means of pressure transducers at a total of two points, in the saturation vessel and upstream of the membrane pore flow reactor, and recorded by the software Labview VI online.

At the outlet of the membrane pore flow reactor, samples can be taken. The quantitative analysis of the reaction mixture is effected by gas chromatography.

### Results

Table 1 shows a comparison of the space-time yields in the catalytic hydrogenation of α-methylstyrene to cumene in various reactor types. The space-time yields of own measurements in the membrane pore flow reactor, in the catalytic fixed bed reactor and in the slurry reactor are compared with published values for trickle film reactors, bubble columns and membrane reactors which work by the diffusor principle. In all studies, a hydrogenation of α-methylstyrene was performed at a temperature of approx. 40° C. and a partial hydrogen pressure of 1 bar with palladium on Al₂O₃ as the support material. Based on the catalyst mass used, the membrane pore flow reactor exhibits the highest space-time yield. The reactor performance of the catalytic fixed bed reactor and of the slurry reactor investigated is higher than the published results for the diffusor membrane reactors, the bubble column and the trickle film reactors.

Based on the volume of the reaction solution, it was possible in the membrane pore flow reactor to achieve similar space-time yields to those in the slurry reactor and in the catalytic fixed bed reactor, since the membrane pore flow reactor has in each case been equipped only with one catalytically active ceramic pore flow membrane. For this reason, the potential determined can be classified as the lower threshold value of the reactor performance. The combination of a plurality of individual catalytically active pore flow membranes to a bundle allows a higher catalyst loading in the membrane pore flow reactor to be achieved, by virtue of which an even higher space-time yield can be realized.

Table 2 lists the change in the space-time yield as a function of the volume flow. This reveals a linear increase in the space-time yield.

Diagrams 1 and 2 show the conversion curves for a membrane pore flow reactor and a fixed bed reactor. As can be seen in the diagrams, in contrast to the fixed bed reactor, stable conversion rates are achieved in the membrane pore flow reactor.

Diagram 3 shows, for the hydrogenation of cyclooctadiene (COD) to cyclooctene, the change in the selectivity as a function of the conversion for various reactor types. This reveals that the membrane flow reactor has a significantly higher selectivity for cyclooctene than the conventional reactor types.

Diagram 4 shows the change in the conversion with time in a membrane pore flow reactor for two membranes with different pore diameters. The diagram shows that the membrane with the smaller pores has a higher conversion rate, which is attributable to better contacting of the liquid with the catalyst particles.

TABLE 1
Comparison of the space-time yield of all reactor types considered in the
hydrogenation of α-methylstyrene

| | | | | STY | STY |
|---|---|---|---|---|---|
| | | p(H₂) | T | [μmol/ | [μmol/ |
| Reactor type | Solvent | [bar] | [° C.] | (s g_{Pd})] | (s l_{reactor})] |
| Diffusor [1] | Mesitylene | 1 | 35 | 8.24 | — |
| Diffusor [1] | Mesitylene | 1 | 50 | 20 | — |
| Diffusor [2] | — | 1 | 50 | 380 | — |
| Intrinsic kinetics | | | | | |
| Slurry reactor [2] | MCH | 1 | 40 | 100 000 | 26 666 |
| Trickle film reactor [3] | Cumene | 1 | 38.2 | 1.11 | 0.12 |
| Trickle film reactor [4] | — | 1 | 40 | 8.64 | 70 |
| Trickle film reactor [5] | Cumene | 1 | 40 | 2.14 | 86 |
| Bubble column [6] | — | 1 | 39.5 | 242 | 714 |
| Own measurements | | | | | |
| Slurry reactor | Cumene | 1 | 40 | 4099 | 274 |
| Fixed bed reactor | Cumene | 1 | 40 | 6851 | 212 |
| Membrane flow reactor | Cumene | 1 | 40 | 8378 | 129 |
| (one membrane) | | | | | |
| Membrane flow reactor | Cumene | 1 | 40 | — | 576 780 |
| (a bundle of 25 membranes) | | | | | |

[1] Cini, Harold, AlChE Journal 37 (1991) 997-1008
[2] Meille, Bellefon, Schweich, Ind. Eng. Chem. Res. 41 (2002) 1711-1715
[3] Babcock, Mejdell, Hougen, AlChE Journal 3 (1957) 366-370
[4] Satterfield, Pelossof, Sherwood, AlChE Journal 15 (1969) 226-234
[5] Turek, Lange, Busch, Löwe, Chem. Tech. 28 (1976) 149-152
[6] Sherwood, Farkas, Chem. Eng. Sci. 21 (1966) 573-581

TABLE 2
Dependence of the space-time yield based on the catalyst mass
on the volume flow
(T = 40° C., p(H₂) = 1 bar, nR = 1600 min⁻¹, 0.35 mol l⁻¹ of
alpha-methylstyrene in n-heptane)
membrane flow reactor: 1.9 μm, mPd = 2.1 mg, VR = 110 ml,
porosity 8.06%.

| Volume flow | Residence time | Space-time yield |
|---|---|---|
| [ml/min] | [s] | [μmols/s/gPd] |
| 153 | 2.20E−02 | 2855 |
| 300 | 1.12E−02 | 5709 |
| 400 | 8.43E−03 | 8012 |
| 500 | 6.75E−03 | 9176 |

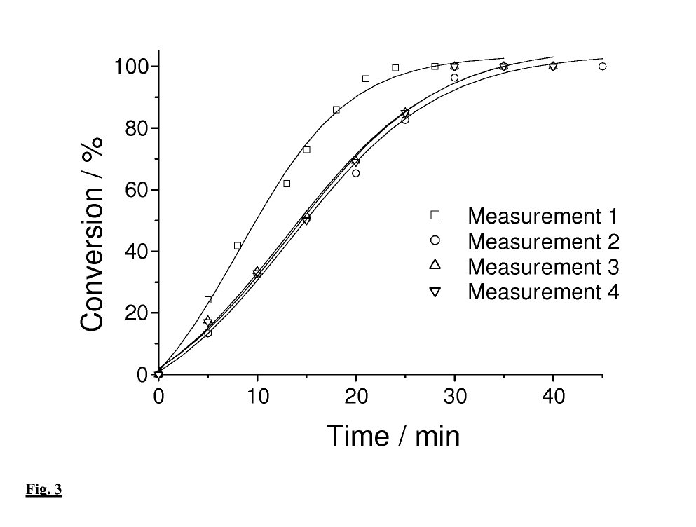

FIG. 3: Conversion curves for alpha-methylstyrene in the flow membrane reactor

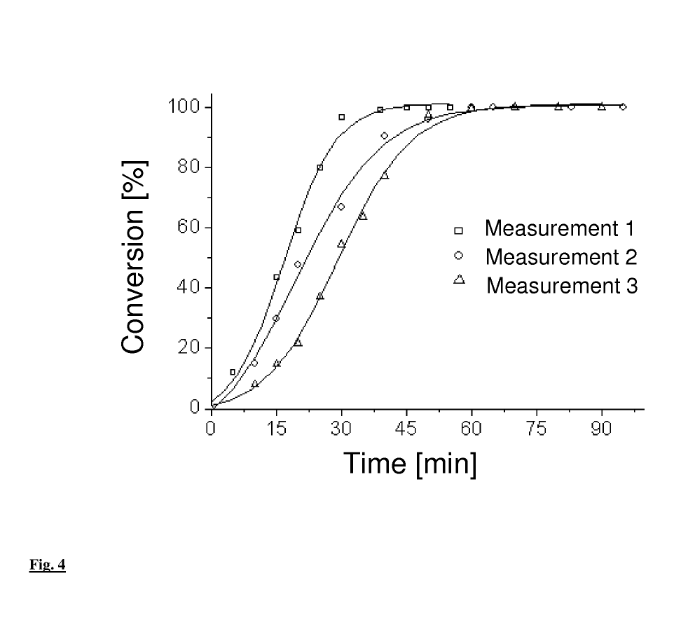

FIG. 4: Conversion curves for alpha-methylstyrene in the fixed bed reactor

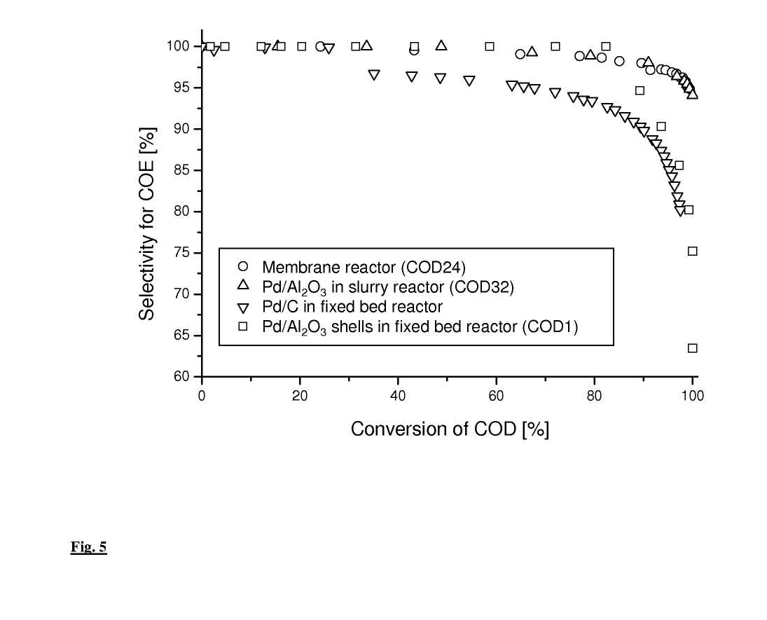

FIG. 5: Comparison of conversion-selectivity profiles in the hydrogenation of cyclooctadiene (COD) (10 bar, 40° C., 10% by volume of COD in heptane, membrane reactor)

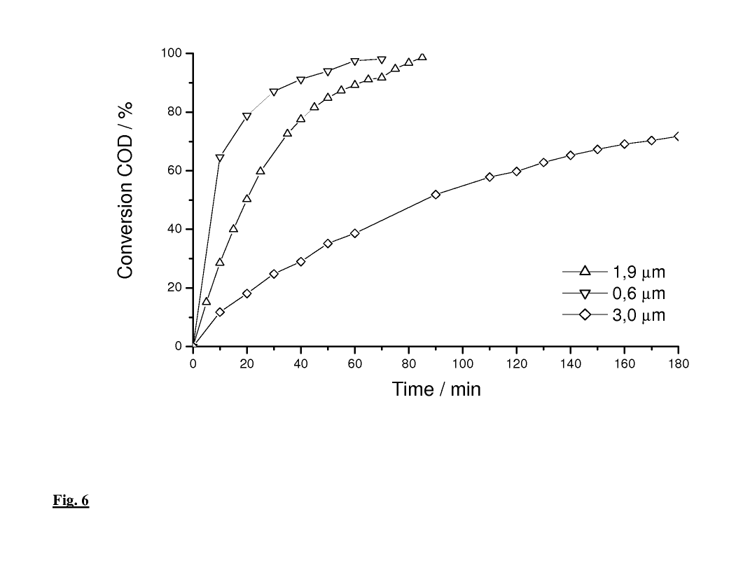

FIG. 6: Conversion curves as a function of the pore diameter for the hydrogenation of cyclooctadiene (COD) in the membrane pore flow reactor (10 bar, 40° C., 10% by volume of COD in heptane)

1. A membrane pore flow reactor with pores occupied by catalytically active species.

2. The membrane pore flow reactor as claimed in claim 1, having pores with diameters in the range of 0.1-10 μm.

3. A process for converting organic compounds, which comprises converting said organic compounds in the membrane pore flow reactor of claim 1.

4. The process as claimed in claim 3, wherein the membrane pore flow reactor has pores with diameters in the range of 0.1-10 μm.

5. The process as claimed in claim 3 wherein the residence time of the reaction solution in the pores is from 1*10−3 to 0.25 s.

6. A ceramic pore flow membrane having a coating of catalytically active components and pore sizes in the range of 0.1-10 μm.

7. The membrane pore flow reactor as claimed in claim 1, wherein said catalytically active species is a catalyst for hydrogenation reactions.

8. The process as claimed in claim 3, wherein said conversion is a hydrogenation.

9. The membrane as claimed in claim 6, wherein said catalytically active components comprise a hydrogenation catalyst.

10. The process as claimed in claim 4 wherein the residence time of the reaction solution in the pores is from 1*10−3 to 0.25 s.
